(19) United States
(12) Patent Application Publication
(10) Pub. No.: US 2014/0236025 A1
(43) Pub. Date: Aug. 21, 2014

(54) Personal Health Monitoring System

(71) Applicant: Michael L. Sheldon, Georgetown, TX (US)

(72) Inventor: Michael L. Sheldon, Georgetown, TX (US)

(21) Appl. No.: 13/908,661

(22) Filed: Jun. 3, 2013

Related U.S. Application Data
(60) Provisional application No. 61/850,507, filed on Feb. 15, 2013.

(51) Int. Cl. G06F 19/00; A61B 5/0205

(52) U.S. Cl. CPC G06F 19/3418; A61B 5/0205
USPC 600/483; 705/3

(57) ABSTRACT

A personal health monitor device comprising memory for collecting and storing attributes from an individual and a processor for quantizing each attribute in such a way as to indicate a normal range for that attribute and for measuring deviations from that normal range. The processor further calculates the well-being of the individual using the deviations measured. The results are displayed indicating the well-being of the individual.

### CROSS-REFERENCE TO RELATED APPLICATION

This application claims the benefit under 35 U.S.C. §119(e) of U.S. Provisional Application Ser. No. 61/850,507, entitled “Personal Health Monitoring System,” filed Feb. 15, 2013, the entire disclosure of which is hereby expressly incorporated by reference herein.

### FIELD OF DISCLOSURE

The present disclosure relates generally to a personal health monitoring system and more particularly to the use of a device or system to monitor a person's physiological condition or attributes and use that information to diagnose and/or advise a user on his or her well-being.

### DESCRIPTION OF RELATED ART

Systems for monitoring various physiological conditions for an individual are fairly common. One such system available today includes a wearable health monitoring system that includes sensors that are integrated with a telemedicine system. For this system, various sensors are attached to an individual and the sensed data that is created is communicated to a phone. Once collected at the phone the data is then sent to a remote server where doctors and trained physicians can analyze the data. Similar types of systems have also been used by athletes for measuring their physical attributes during training. Again, these systems collect the sensed data and then send the information to a tablet or computer to analyze it. Sometimes a phone is used to get the data to the tablet or computer. In these type of systems, performance is measured, not the health and well-being of the user.

What is needed is a health monitoring system that is integrated into a cellular phone or a tablet having cellular or internet communications which allows a user to collect a wide variety of data, including various physiological conditions, and to analyze the data for the purpose of determining the well-being of that user. Then should the need arise, the collected data, analysis, or other information could be sent to a treating physician using the cellular communication feature for further evaluation. This type of system would not only be convenient and practical, since everyone is currently using their cell phone or tablets for a variety of other application, but also beneficial because it would allow the user to maintain control and security over that personal individual data. As a result, great deal of expense in time and money could be saved by avoiding unnecessary doctor visits.

### SUMMARY

This invention relates to a personal health monitor device which may be a cellular phone or tablet with internet connection. The device includes a memory for collecting and storing attributes from an individual. The device further includes a processor for quantizing each attribute in such a way as to indicate a base line and a normal range for that attribute. Once the base line and normal range has been identified deviations from the base line and/or normal range are identified. These deviations are then used to indicate possible symptoms indicating the well-being of the individual. These symptoms are compared to symptoms of known illnesses to determine if the individual may have a known illness. The results of this comparison are displayed to the individual on a display. Should the individual wish to send the results to trained medical personal, he or she may transmit the results or any attributes which led to those results using internet or cellular communications.

### BRIEF DESCRIPTION OF THE DRAWINGS

FIG. 1 is a diagram of a personal health monitoring system capable of collecting and storing data representing a user's personal attributes from different sources for the purpose of analyzing the attributes using a processor to determine and display the well-being results of the user.

FIG. 2 illustrates a display of a phone or tablet showing functions that may be selected for monitoring the health of a user.

FIG. 3 shows an example of a series of health assessment questions that the monitoring device could query the user for to collect additional information regarding the user's weight and blood pressure and some calculated results such as body mass index and general assessment of blood pressure based on those answers.

FIG. 4 shows an example of a series of questions used to query the user regarding medical history of the user.

FIG. 5 illustrates another display of a phone or tablet showing functions that may be selected for monitoring the health of a user.

FIG. 6 shows a graph illustrating the measurements of an attribute such as blood pressure for the user over a 24 hours period.

FIG. 7A shows a graph illustrating the measurements of the base line and trend of an attribute such as blood pressure of the user over a period of 30 days.

FIG. 7B shows a graph illustrating the measurements of the base line trend of an attribute such as blood pressure of the user over a period of 5 years showing the effectiveness of medication taken by the user to lower the user's blood pressure.

FIG. 8 illustrates another display of a phone or tablet showing suggestions generated by the health monitoring system that the user may want to consider as a result of the historical analysis of the users blood pressure.

FIG. 9 is flowchart illustrating the process of analyzing the measured attributes of the user to determine the well-being of the user.

### DETAILED DESCRIPTION

The present invention relates to an individual health monitoring system that is preferably integrated into a cellular phone for personal use. While a cellular phone is shown and described as the preferred embodiment, a tablet or similar computing device that has cellular communications capabilities or internet access is contemplated as a part of the concept of this invention. According to the concept of this invention, the phone or tablet includes or has access to a large data base for storing all of the data collected from sensing devices used to collect physical attributes of the individual and software applications that can be called upon by the user to evaluate the collected data to determine the well-being of the individual. Results of the analysis, suggestions, or recommendations for improving that individual's health would be displayed on the phone's or tablet's display screen. Depending on the results of the analysis, the user may select to send these results and/or data used to generate these result to a treating physician using standard cellular communication or internet access features available today. Further, informational data regarding prescribed drugs taken by the user and their known side effects are preferably stored in the data base or may be accessed or retrieved using the cellular or internet connection. As a preferred feature of the system the physical attributes of the individual that are monitored may also be used to determine if any undesirable side effects exist as a result of taking these medications so that an alert can be provided to the user.

Preferably the data is collected wirelessly from the sensing devices, but it could be downloaded directly from the sensing devices using typical wired connections such as USB cables. The system could be used in a continuous mode for collecting individual data or uploaded periodically from a sensing device which is capable of collecting and storing the information. Information may also be manually entered in by the user. Typical questioners often provided by doctors and required to be filled out by a user could be used as a means for collecting personal information.

The data base used for storing this information must be large enough for storing large amounts of data regarding the physical attributes of the individual and includes at least one, but preferably may include many such physical attributes as body temperature, blood pressure, humidity, ECG, breathing, blood sugar, heartbeat, administered medications, past medications, etc. Preferably the data is stored in association with the time that the data was created to provide a time line for the data. This would allow for historical time lines of data to be evaluated as well as identify and notify the individual when fresh data is needed to properly analyze the well-being of the individual. Using this data, normal base line and normal range data that is unique for that person can be identified. The normal base line for a particular attribute could be calculated as the average measurement for the day, the average measurement over a period of 30 days or any medical acceptable range for a given attribute. An acceptable deviation or range from that base line could vary depending on what would be considered normal for that particular attribute. For example, not every one's average body temperature is the same. As appreciated by those skilled in the art, what may be a normal base line or range for one person is not necessarily the normal base line or range for another. The same would apply to blood pressure, heart rate, breathing, blood sugar, etc. Still further, trends for the base line and ranges can be detected and analyzed. For example, depending on the time of day or as some of us age our blood pressure tends to trend in different directions. In the morning our blood pressure is usually highest. As we age, our blood pressure trends upwards, especially if we have a family history for high blood pressure. Understanding these normal ranges and trends can be very important for diagnosing a person's well-being as well as understanding how to properly prescribing medication if needed. Plus, by monitoring the base-line, ranges, and the trends, the user (and the doctor) can monitor the effectiveness of medication. Further, as would be appreciated by those skilled in the art, other applications are possible. For example, this health monitoring system could be used for those that work in toxic environments. Health effects related to exposure to those toxins could be monitored.

It is also preferred that additional information could be stored to further aid in the analysis of a person's well-being by including information such as medical history including if possible family medical history and personal medical history. Additional information that is unique to the individual could also be entered by the individual to form a more complete data set. For example information on food consumption, types of food, exercise information, sleep information, weight, prescription/medication (current and past), etc. It would be obvious to one skilled in the art that this information would be formatted in such a way as to allow it to be easy accessed and read as necessary by an application analyzing the data as well as be fully searchable. For example, being able to search and review current and past prescriptions can be critically important to determine the compatibility of new medication.

One of the obvious benefits of such a system is that using the artificial intelligence of the system, the system could discover, query the individual, or automatically identify symptoms, rather than asking the individual to recognize the systems for himself when a well-being application is selected. Often times an individual doesn't understand or appreciate what symptoms that they should identify as important. Another advantage is that the system could effectively be operated as a personal doctor's aid by providing medical alerts or early detection of diseases or harmful conditions which could even include reactions to current or new medications. It could also detect when the user missed taking prescribed medications and alert the user. In general, the system would enable an individual to monitor his or her own health and only consult a doctor if the need exists. Many unnecessary doctor visits could be eliminated. Further, by centralizing all the data on a personal device such as a cellular phone or tablet, the information can be keep confidential, secure, and under the control of the individual. If that individual wishes to share that information, the cellular or internet communications feature provides a convenient way to share information or data with a treating physician. For example, imagine a patient that has high blood pressure and his prescription is about to run out. Rather than making an appointment to get a refill, the patient monitors his own blood pressure with a device approved by his physician and then sends the data to the physician who can then approve the proper dosage and renew or change the prescription at the pharmacy directly. Both the physician and the patient save a considerable amount of time which results in cost savings to the patient. This is just one example of the utility and benefit of such a device/system described herein. One skilled in the art would appreciate or could envision many other such examples of savings of time and money in the health care industry that could be employed using such a device/system.

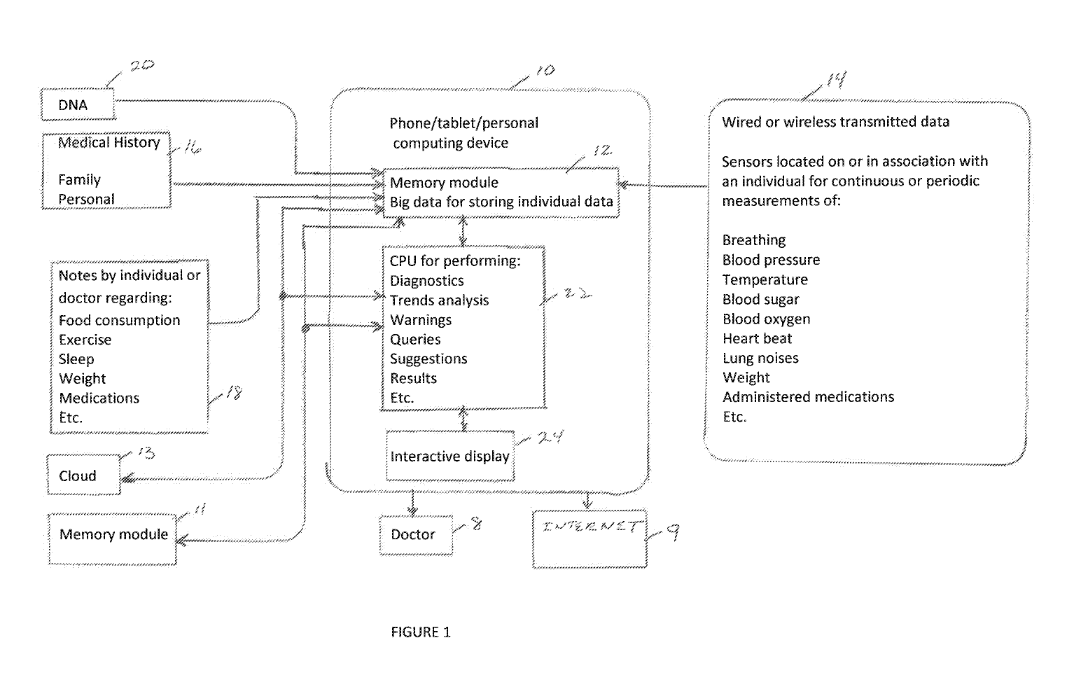

Referring now to the FIG. 1, a schematic diagram of a phone 10 is shown with data being communicated to it from various sources. As mentioned above, it is contemplated that a tablet or similar computing and display device could be used instead of a phone. It is preferred that such a device have a cellular communications feature to allow sharing the collected information with a treating physician 8 or to access or to retrieve data using the internet connection 9. As shown, the phone 10 includes a high capacity memory module 12 for storing large amounts of individual data. Preferably the data is time stamped or references a time when the data was actually collected. While FIG. 1 shows a memory module for storing the individual data, one skilled in the art would appreciate that this data could be stored using a flash card, Sims card, or a similarly high capacity memory 11 connected to the phone or tablet 10. Further, the data could be remotely stored using the Cloud 13 technology which is commonly used to store data using conventional cellular or internet technology and then called upon by the software application selected by the user to evaluate the collected data to determine the well-being of the individual. Alternatively, the artificial intelligence for evaluating the data collected could be stored at the cloud 13 where it can periodically process the data and send the results of such evaluation to the user's phone or tablet 10. To facilitate storage of large data, there are all kinds of data compression techniques available that could also be used to aid in the storage of such large amounts of data and are well known by those skilled in the art. For example, loss less compression and lossy less compression techniques can be used and have long been used by those skilled in the art to avoid storing unnecessary information and thereby increasing the capacity of the memory available. Similarly, there are other techniques to help in extrapolating data when data is missing in the time line and may be used as an aid to analyze the current data for the well-being of the individual as would be appreciated by one skilled in the art.

Data is collected from at least one sensor and preferably several sensors 14 used to collect data in association with the physiological condition or attributes of the individual. The more types of information over time that is collected the better the opportunity to data mine it for creating base-lines, ranges, trends, diagnostics, prognostics, etc. For example, breathing, blood pressure, temperature, cholesterol, blood sugar, blood oxygen, heart beats, lung noises, weight, administered medications, etc. are some of the parameters contemplated. Many others are possible as would be appreciated by one skilled in the art. The sensors can be located on or in association with the individual user by way of a vest, armband, wrist band, ankle band, etc. Many of these devices are readily available. For example, Best Buy currently advertises and sells a host of wireless devices that sense and monitor individual physiological conditions. One such device is a wireless activity and sleep tracker. Another device is a “BodyMedia—Fit Link Armband” that measures calories burned, body temperature, steps and sweat, sleep quality, etc. and is wireless. Still others sell monitors for measuring blood pressure and blood sugar. The sensors could also be intrusive to the individual such as pace makers or other devices which may for example deliver medication to the individual. In the preferred embodiment the data is collected by the sensing device and transmitted wirelessly to the data storage module (11, 12, and/or 13). However, data may be communicated from the devices to the data module in the phone or tablet 10 via wires, such as a USB cable. In each case, time associated with when the data is collected is stored.

Since each individual is unique, relevant data from other sources are also preferred. As shown in FIG. 1, data including medical history for both family and personal 16 are also preferred. Additional information by the individual may also be provided. For example, information regarding the individual's food consumption, exercise routine, sleep habits, weight, medications, etc. 18 may also be provided. DNA information 20 may also be important information in the future for properly analyzing the data and could be included. Taking samples of bacteria from different areas of the body for analysis is also contemplated. As well-being programs are developed, one skilled in the art would realize that other data may be needed and collected. In other words, as the artificial intelligence for analyzing the data improves to detect the well-being of a person, additional data or data collected in a new way may be necessary to complete the analyzes and is contemplated by the concept of this invention.

In the preferred embodiment, the processor 22 of the phone or tablet 10 is used to initiate applications which access the data from the memory module (11 and/or 12) or the Cloud 13 and perform calculations using the collected data to assess the health or well-being of the user depending on the health or well-being programs selected or continuously operating in the background monitoring the user's health. However, it is possible for a second processor to be provided and dedicated to these applications. As an example of the type of applications that could be provided, one could include a general diagnostics application on the well-being of the individual's blood pressure to determine if there may be issues of high blood pressure indicative of heart attacks, strokes, heart failure, kidney disease, stress, etc. Similarly an application could be provided to assess the individual's blood sugar to identify issues with diabetes. Since your blood sugar can fluctuate throughout the day, understanding your sugar levels over time will be important. Trends regarding the above conditions as well as various other health conditions such as good and bad cholesterol could also be performed. General health assessments, warnings, suggestions, and recommendations are a few of the benefits of these applications. It should be understood by one skilled in the art that, while examples of blood pressure and sugar levels are monitored, all of the data from a variety of different data sets (blood pressure, temperature, heart rate, etc.) could and should be used in the analysis and diagnostics of the individual. In other words, analysis of the individual is not limited to looking at just the data set for a given personal attribute. Numerous other medical applications to assess the individual's well-being are also possible but not mentioned here, because it would be obvious to one skilled the art that many such applications could be developed and downloaded on the phone or tablet and used in the manner described above. The resulting analysis and interaction with these applications can be shown on an interactive display 24 that is commonly available on the phone or tablet 10 and may be shown in many useful and creative ways. Graphs or tables showing normal ranges, base lines, trends, or statistics showing data are possible. Colorful alerts, warnings or suggestions may also be displayed. Sound alarms are also possible. Further, forms can be created and displayed for querying the individual for more information to complete the data set for analysis. It should become clear that the concept of this invention contemplates storing and allowing the user to access many different expert medical applications which leverages the wealth of medical knowledge to evaluate and diagnose the data to determine the well-being of the individual.

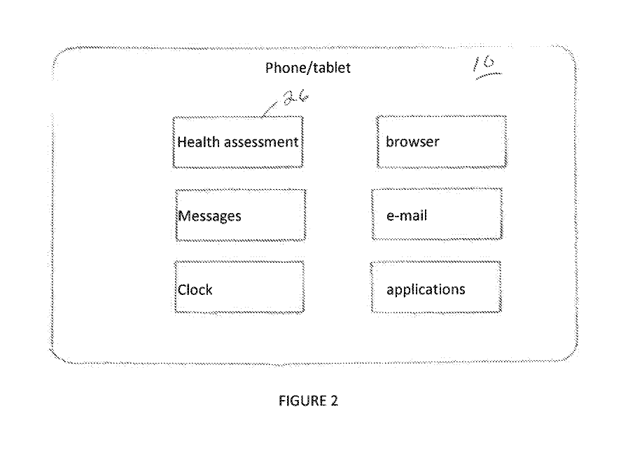

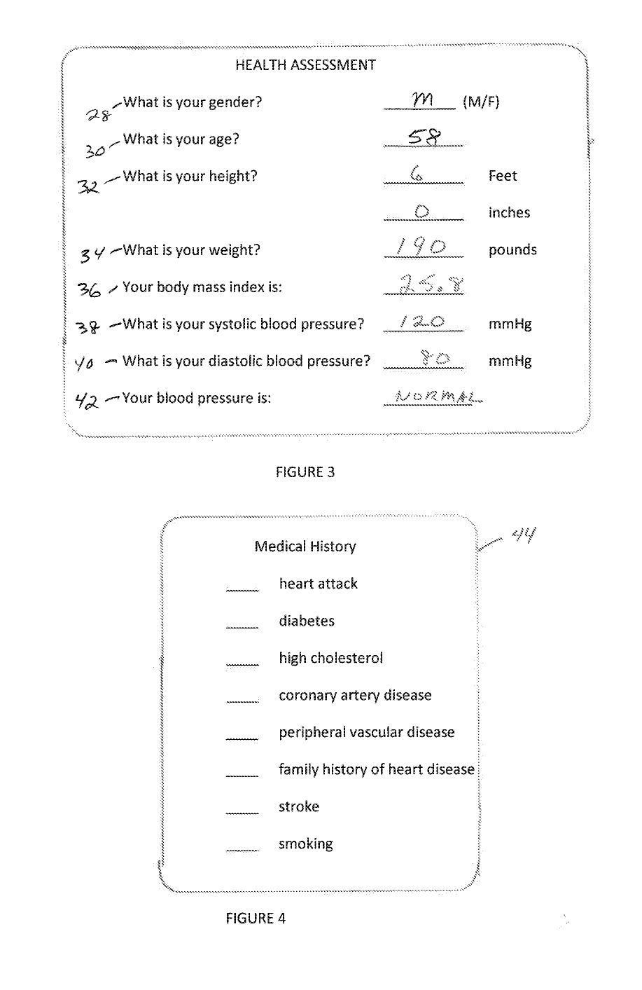

Referring to FIGS. 2-7, some screen shots of a cellular phone or tablet 10 are shown and are used to illustrate how a user might: select one of several potential health assessment applications that could be downloaded and stored on his phone, be queried to enter information, and see displays showing the results of the analysis of the assessment. One skilled in the art would understand that many other types of applications could be stored and selected by a user, questions could be asked of the user to help determine the well-being of the user, and displays showing different parameters, trends, etc. could be shown. As one example of an application that could be stored and selected, FIG. 2 shows a general health assessment program 26 using the interactive screen of his phone or tablet. Selecting this application could result in a query for information from the user. As shown and illustrated in FIG. 3 that could include the user's gender 28, age 30, height 32, and weight 34. This information could then be used to calculate the user's body mass index 36. Additional information could be queried such as blood pressure including the systolic and diastolic values 38 & 40. Based on this information, a health indication 42 can be automatically displayed as high, normal or low blood pressure to indicate the well-being of an individual. While this example demonstrates how a user would be queried to enter information regarding the user's blood pressure measured results, this information could be transmitted to the phone wirelessly or by wire from the measuring device as suggested above. As already mentioned and appreciated by one skilled in the art, a tablet or like device could be used in place of the phone for performing the general health assessment.

Referring now to FIG. 4, quires could also be made for medical history. As an example, a form 44 is shown asking the user to provide information on his medical history by interactively placing a check next to the appropriate items shown. One skilled in the art would appreciate that there is an endless amount of medical history information that could be obtain from the user to help in the medical assessment of the well-being of the user. These forms could be arranged and appear in an endless variety of ways. The present form shows spaces that can be checked that would allow the user to select past medical issues or current conditions that would apply to that individual, such as previous heart attacks, diabetes, high cholesterol, coronary artery disease, peripheral vascular disease, family history of heart disease, stroke, and smoking just to name a few that are possible.

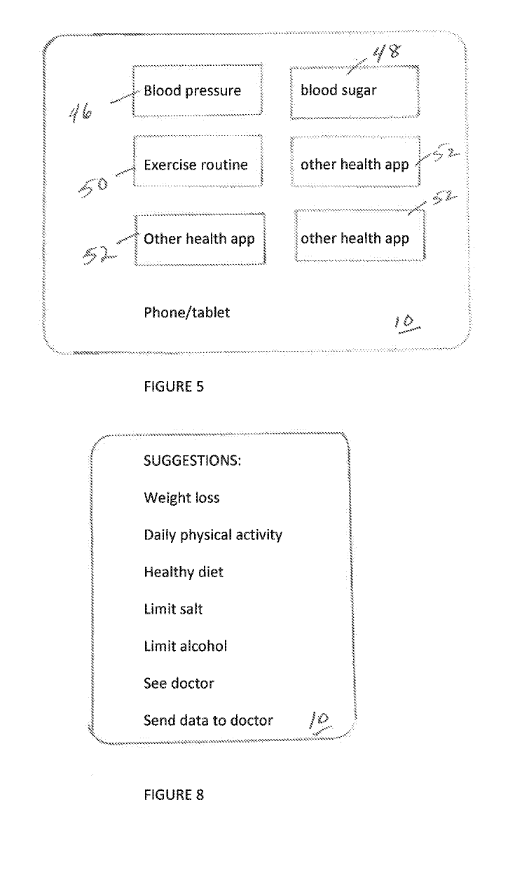

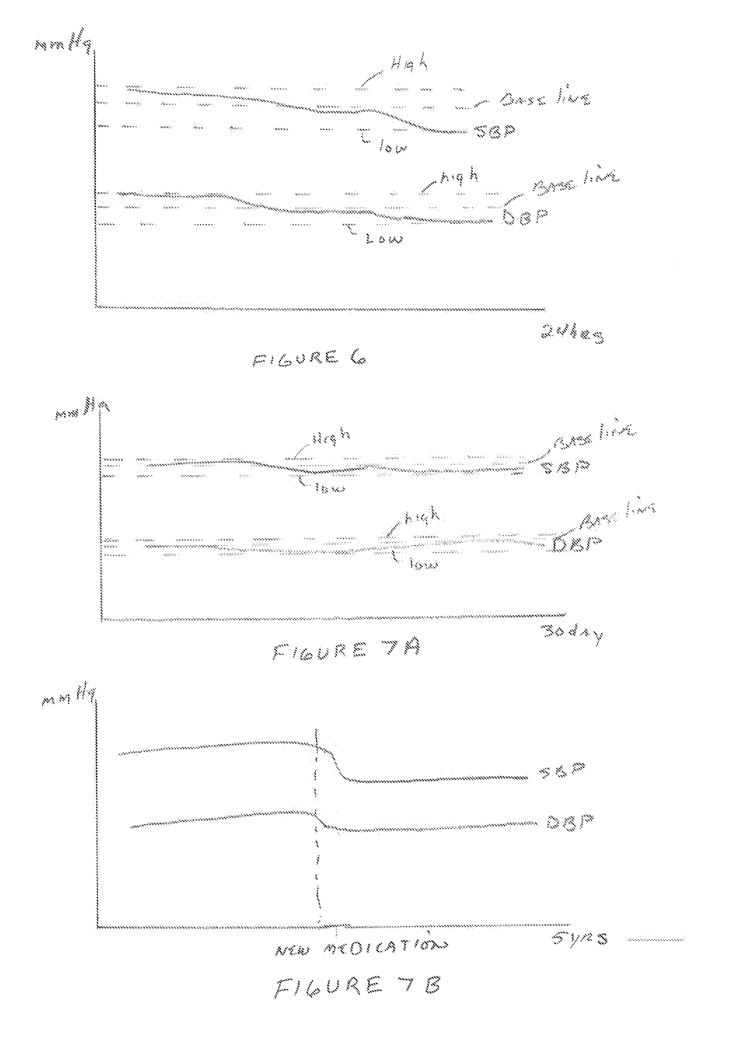

Turning the attention of the reader to FIG. 5, the user could select applications from his main menu shown of his phone or tablet 10 shown in FIG. 2 to display a variety of health monitor applications such as blood pressure 46, blood sugar 48, exercise routine 50, and others 52 as shown. As one skilled in the art would appreciate, there are a variety of health monitor applications that could be created, stored and selected by the user. The applications shown are merely examples of the types of health application that could be installed and selected by a user. Many others are possible and contemplated by the present invention. As an illustration, the user could select the blood pressure application. Based on the historical information on measured blood pressure a graph could be displayed showing the user his blood pressure over the last 24 hours as illustrated in FIG. 6, over the last 30 days as illustrated in FIG. 7A, or over any other desired range. FIG. 7B is provided to illustrate the results and effectiveness of a user that has taken medication to reduce his blood pressure. In the alternative, it should be appreciated by one skilled in the art that the health device/system could be used to measure the effects of a user exposed to chemicals in a toxic environment. The attributes of the user can similarly be monitored.

Rather than showing a graph as shown, tables or other ways of showing this information are possible and would be appreciated by one skilled in the art. Depending on the results of the blood pressure data, it is preferred that suggestions are provided to the user for improving the results of the users blood pressure as illustrated in FIG. 8. This could include such things as a recommendation that the user lose some weight, increase his daily physical activity, improve the user's diet (with recommendations of the types of foods that the user should avoid or include in their diet), limit his salt or alcohol intake or see a doctor regarding his blood pressure or medication therefor. A variety of other suggestions or recommendations is possible and would be realized by one skilled in the art depending on the data collected and analyzed. Further, the health monitor application could further refine these recommendation or suggestions by using the historical data collected from other measured physiological attributes of the individual. For example, the application could eliminate some of these suggestions or recommendations to one or two for the user to consider and follow. Therein lays one of the benefits of this concept. By collecting data uniquely from one individual, the recommendations or suggestion can be tailored, based on that data, to the needs of that individual. For example, maybe the user has a healthy diet and is in great physical shape. The only recommendation may be that they consult their doctor. If the only recommendation or suggestion is to see your physician, then the information that resulted in this conclusion could be sent to the physician using email or internet features common to most cellular phones or tablets.

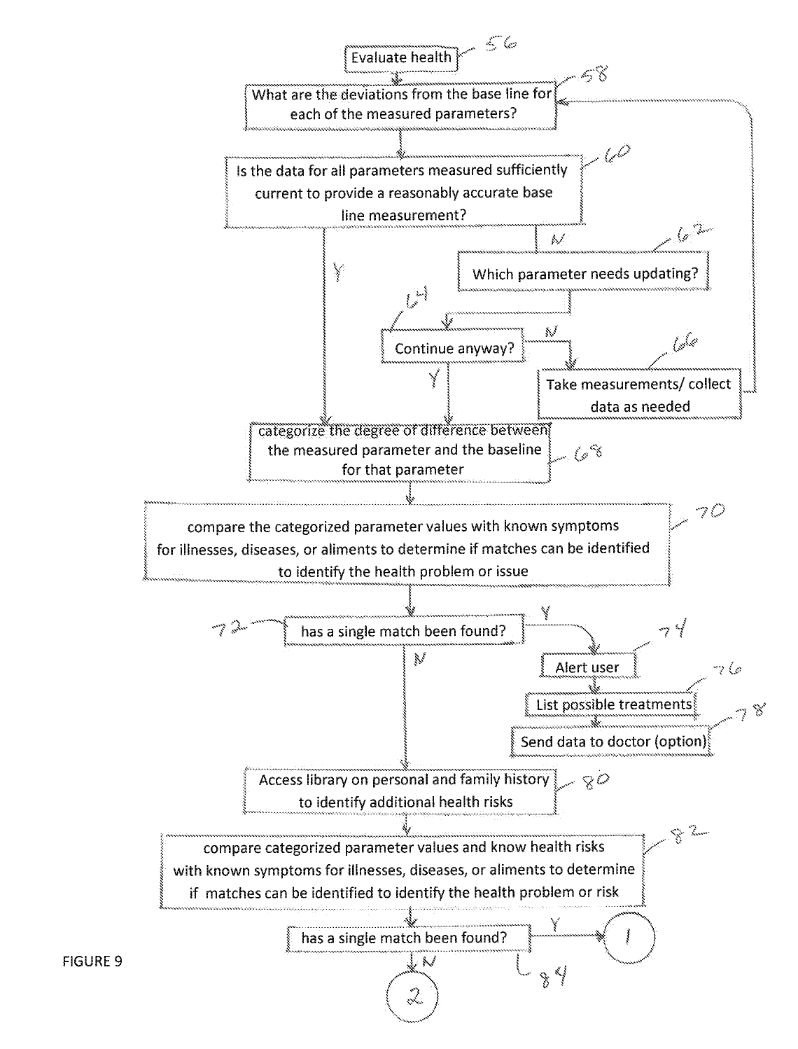

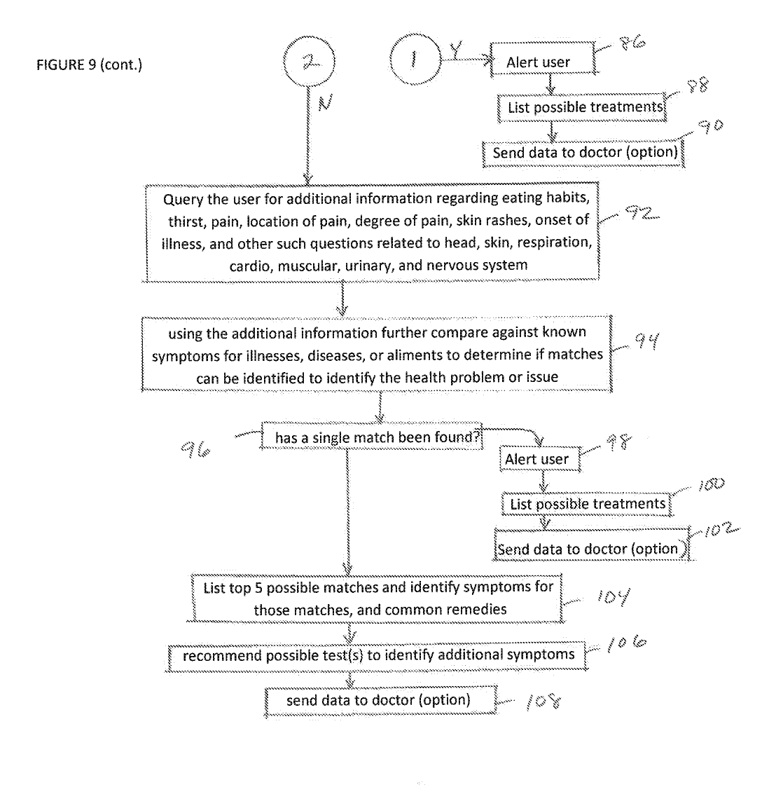

Referring now to FIG. 9, the following illustrates how such a system might analyze the information to show how the health monitoring system can narrow down the possible causes of an ailment or diagnose the ailment. To start, the user can select the health assessment button 56 to start the analysis. The first step in the process is to look at the measured parameters and identify deviations from the base line or range for each of the measured parameters 58. A determination is made to see if the data is current and sufficient to provide a reasonably accurate base line and range measurement. For this example, it is assumed that there is a sufficient collection of data over a period of time to determine a reliable normal basis and range for each parameter measured. It is also preferred that there is sufficient data to provide a trend for these parameters too. The actual amount of historical data needed to provide a reliable trend, base line, and range measurement may depend on the parameter measured. Alternatively, medically acceptable base lines and ranges could be used. The system preferably has default which would recognize when the measured information is sufficiently current enough to be used in any evaluation 60. If not, then the attribute that needs updating is identified 62 and a decision is made by the user to either continue or to collect the necessary data before proceeding 64, 66. For example, if blood pressure has not been taken for a period of a couple of months, then a recommendation could be made to the user to take several blood pressure readings over the next couple of days to provide more current data for the evaluation. If one of the parameters is cholesterol, then data collected every couple of months may be more than enough to provide a reasonable amount of data for determining a base line measurement. In the preferred embodiment the system would recognize when new data is needed to update the system with reliable data for evaluating health before the user even requests a health evaluation and sends the user a message or puts the user on notice that new data is needed. The system could account for this choice by providing a waited value for these parameters when they are not current.

As shown, the user has a choice to proceed with the analysis with slightly outdated data or no data at all. Medically acceptable ranges such as 120 over 80 for blood pressure could be used to complete the analysis when there is no data, the data is incomplete or when it is outdated. For the cases where the user decides to continue with the analysis, it is preferred that at the end of the analysis, recommendations are provided to the user to collect more data regarding certain parameters for a more accurate analysis.

Deviations from the base line and/or the normal range can be classified or categorized by identifying the deviation as normal, a little high, high, a little low, low or by scaling the deviations placing a scaling value such as 1 to 10 between normal and high and similarly −1 to −10 between normal and low 68. Other ways of scaling, classifying, or categorizing the deviations from normal are possible and would be obvious to one skilled in the art. For the present example, let's assume that the currently measured parameters indicate that blood pressure is a low, temperature is normal, heart rate is a little high, respiratory is normal, weight is a little low, Oxygen saturation level is normal, and glucose is high. At this point the system could access a library of know illnesses, diseases, or aliments to compare their known symptoms to the identified categorize parameters to identify possible matches for candidates that may be causing the user to have poor health 70. If the list of possibilities is significant, more investigation is necessary 72. Typically with a limited number of parameters measured, more information will be need. If however a match is found, the user can be alerted 74 and a list of possible treatments could be provided 76. Alternatively, this information including the data and the results can be sent to a doctor 78. If no match is found, more information is needed to complete the analysis.

Next the library of information on personal and family history is accessed 80. Such things as medications that the user is taking and its possible side effects are considered to determine if such side effects would result in some or all of the conditions indicated by the measured parameters 82. Similarly other historical conditions are considered such as race, gender, age, past medical history, prior illnesses, previous surgeries, alcohol usage, smoking habits, exercise activity, dietary, allergies, etc. For the present example, let's assume that the user has a history of being overweight and his glucose trend over the past year has been running on the high side. Based on the previous analyses, these factors appear to be significant factors when combined with the measured parameters and then compared with known symptoms of known illnesses, diseases, or aliments 84. If a match is identified, the user is alerted 86 and a list of possible treatments could be provided 88. Again, the user has the option to send all of the information to his doctor 90. If no match is found, more information will be needed to continue with the analysis.

To help narrow down the illness, a list of questions is preferably asked of the user 92. These questions are the typical question that you would see at a doctor's office on your first visit for an illness. They can include such things as is there any pain? Where is the location of the pain? What is the degree of pain on a scale of 1 to 10? Are there any skin rashes? Did the illness onset come quickly or slowly? Is there congestion? Is there a cough, head ache, tired, restless, etc.? Generally the typical questions are directed to the head, skin, respiration, cardio, muscular, urinary, and nervous system. For the present example the user has noticed an increase in the need to urinate.

Based on this line of questions along with the current parameters measurements, the personal and family history and the questions, a preliminary diagnosis might be determined and recommendations made or further questions asked 94. For example, questions regarding whether the user has been eating normal, has there been excessive hunger, excessive thirst, pain, etc. Once these questions have been answered by the user, a determination is made as to whether there is a match in symptoms 96. For the present case let's assume that there was excessive hunger and thirst. These symptoms, when combined with the above help narrow the analysis and would suggest that the health issue may be related to diabetes, urinary tract infection, or other disorders that may require the attention of a doctor. The user is alerted 98 and possible treatments are identified 100. The analysis and the basis of this diagnosis could be downloaded and then sent to your doctor using the email or internet features of your phone or tablet 102. If the diagnosis were to be something less threating, such as a cold or flu, common remedies or over the counter medications might be suggested. In all cases it is a preferred embodiment that the health system identifies the possible causes for health problems, the symptoms of those causes, and/or the list of possible treatments. In the case where no match has been found, several of the closest matches, for example the top five matches along with their symptoms and common remedies could be brought to the attention of the user 104. Further, recommendations on the type of tests that could help identify the illness could be displayed to the user 106. All of these results can then be sent to the doctor 108.

It should be obvious to one skilled in the art that the above example is only illustrative and that this personal monitor health system is not limited to finding or diagnosing illnesses but also looking for side effects of prescription and non-prescription medications. It could also look for conflicts or the effects of combining medications and alert the user. It is preferred that the data module include a library that contain at least a list of known side effects of medications that the user is taking so that it can be compared to the measured parameters to look for these side effect and to ask questions of the user for more information should some of these side effects be detected. For example, questions similar to those above or directed specifically to the indicated side effects of the medication. If the issue relates to a possible reaction to a current medication that the user is taking, an alert is given to the user along with the known side effect of that medication. The user can thereafter send this information to his or her doctor using email or internet capabilities.

While the invention has been particularly shown and described with reference to a preferred embodiment, it will be understood by those skilled in the art that various changes in form and detail may be made therein without departing from the spirit and scope of the invention. For example, it would be understood by those skilled in the art that accumulating large amounts of data from an ever increasing list of individual parameters that can be measured over time could result in a significant improvement in the results of any analysis. Further it should be appreciated by one skilled in the art that the flow diagram shown in FIG. 9 is only illustrative of determining a user's health. There are a host of mathematical tools available that could be used in analyzing the data such as principal component analysis which could be performed on the data to identify contributing parameters (attributes) that resulted in certain illnesses. As one skilled in the art would appreciate, principal component analysis can be used to take a look at the raw data from various parameters to determine what the important contributors were that caused that particular result. This analysis can be running in the back ground while data is being collected and called upon by the user. Once the important contributing parameters are identified, the principal component analysis can then be directed to the finer more limited set of parameters to validate the analysis. In situations where you have sufficient data with regard to that user, you can start with the problem, disease, illness, or aliment and then using this analysis look for contributing causes. As one skilled in the art would appreciate, there are many more mathematical tools available that can similarly be adapted and used on the collected data to discover root causes and effects of various conditions of the user. For example, various mathematical models are currently being used in the process control industry and can similarly be adapted and used to predict the oncoming of certain conditions such as cold sores, colds, heart disease, diabetes, etc.

What is claimed:

1. A method for monitoring the well-being of an individual comprising the steps:
collecting data from at least one sensor detecting physical attributes of an individual in memory of a cellular phone, wherein the data represents the physical attributes,
storing at least one well-being software application in the memory of the cellular phone for analyzing the data to determine the well-being of the individual,
selectively running the software application to analyze and determine the well-being of an individual, and
displaying at least one of the results of the analysis or determination of the well-being of the individual.

2. A personal health monitoring device comprising:
memory module capable of storing data representing multiple attributes of an individual, wherein the data has time associated with it to identify when the data was collected,
at least one well-being software application selectable by a user for assessing the well-being of the user,
a processor that is capable of accessing the memory module for data to analyze the well-being of the individual in accordance with the well-being software application selected by the user and providing results indicating the current well-being state of the user, and a display for displaying the results of the analysis to the user.

3. The personal health monitoring device of claim 2 further includes at least one of cellular or internet communications.

4. The personal health monitoring device of claim 2 wherein the device is a cellular phone.

5. The personal health monitoring device of claim 2 wherein the device is a tablet.

6. A personal health monitor device comprising:
memory for collecting and storing data indicative of the physiological attributes of an individual;
a processor for quantizing the data indicative of each attribute in such a way as to indicate a normal range for that attribute and for identifying deviations from that normal range;
wherein the processor further calculates the well-being of the individual using the identified deviations measured; and
a display for displaying the well-being of the individual.

7. The personal health monitor of claim 6 wherein the device is a cellular phone.

8. The personal health monitor of claim 6 wherein the device includes communication means for transmitting the calculated results of the well-being to a physician.

9. The personal health monitor of claim 6 wherein the device is a personal computing device that further includes communications means for transmitting the calculated results of the well-being to a physician.

10. The personal health monitor of claim 6 wherein the device is a cellular phone.

11. The personal health monitor of claim 6 wherein the processor determines trends for each attribute.

12. The personal health monitor of claim 6 wherein the processor determines a base line for each attribute.

13. The personal health monitor of claim 6 wherein the device is used to measure the effects of medications taken by the individual.

14. The personal health monitor of claim 6:
wherein the measured deviations indicate symptoms of the individual and the device further includes a library of illnesses and symptoms for each illness, and
wherein the processor compares the symptoms for the individual with the symptoms for each illness to determine if the individual may have an illness with the same or similar symptoms.

15. The personal health monitor of claim 14 further including an alarm that warns the individual if an illness with the same or similar symptom is detected.

16. The personal health monitor of claim 6:
wherein the identified deviations indicate symptoms of the individual and the device further includes a library of side effect symptoms for each medication that the individual is taking, and
wherein the processor compares the symptoms for the individual with the symptoms indicating the side effect symptoms for each medication to determine if the individual may have an adverse reaction to the medication.

17. The personal health monitor of claim 16 further including an alarm that warns the individual if an adverse reaction to the medication is detected.

18. The personal health monitor of claim 6 wherein the well-being software application includes measuring and monitoring blood pressure.

19. The personal health monitor of claim 6 wherein the well-being software application includes measuring and monitoring cholesterol.

20. The personal health monitor of claim 6 wherein the memory includes the use of cloud technology.
